(19) United States
(12) Patent Application Publication
(10) Pub. No.: US 2008/0138907 A1
(43) Pub. Date: Jun. 12, 2008

(54) Extractant Solution for Residual Veterinary Agent in Livestock Product or Seafood

(75) Inventors: Masahiko Kitayama, Bloomington, IN (US); Masaki Kosono, Ibaraki (JP); Takashi Omori, Ibaraki (JP); Fumiki Morimatsu, Ibaraki (JP); Ryoji Yamada, Ibaraki (JP)

Correspondence Address: BIRCH STEWART KOLASCH & BIRCH, PO BOX 747, FALLS CHURCH, VA, 22040-0747, US

(73) Assignee: Nippon Meat Packers, Inc., Osaka-shi (JP)

(21) Appl. No.: 11/793,135

(22) PCT Filed: Dec. 16, 2005
(86) PCT No.: PCT/JP05/23557

§ 371 Date: Sep. 17, 2007

(30) Foreign Application Priority Data
Dec. 16, 2004 (JP) 2004-365135

(51) Int. Cl. G01N 33/12

(52) U.S. Cl. 436/17

(57) ABSTRACT

Disclosed is an extracting solution for use in the extraction of a residual veterinary drug in livestock product or seafood. The extracting solution contains 0.05 to 1% of metaphosphoric acid and comprises a buffer solution of pH 5.5 to 7.5. The extracting solution can extract plural veterinary drugs at the same time, and can be used preferably used for the extraction when the residual veterinary drug is an antibacterial substance. By using the extracting solution, it becomes possible to extract plural veterinary drugs efficiently at the same time without impairment of stability because of its neutral pH range. Hence, the extracting solution enables to determine a residual veterinary drug in livestock product or seafood rapidly in a simple manner.

### TECHNICAL FIELD

The invention relates to an extracting solution for residual veterinary drug. More specifically the invention relates to the extracting solution used for measurement of veterinary drug remaining in livestock product or seafood.

### BACKGROUND ART

Recently, various veterinary drugs are used in animals for prevention and treatment of diseases and promotion of growth. Measurement (and determination) of the residual level of veterinary drugs in livestock product or seafood is very important for assuring safety of food. In this situation, throughout the world, as a result of introduction of positive list system of veterinary drugs remaining in livestock product and seafood, provisional maximum residue limits of more than 200 veterinary drugs are specified, but it is impossible to individually analyze the drugs by the known methods in terms of time and labor. Therefore, it is urgently needed to establish a method that can analyze multiple drugs simultaneously, quickly and simply.

Veterinary drugs, in particular, antibacterial substances (such as antibiotics and synthetic antibacterial drugs) are widely used both in variety and quantity in the present mass rearing trend, and possibility of residue in livestock product and seafood is feared.

However, with veterinary drugs presently approved by the Japanese Food Hygiene Law, useful technique is not known yet for simultaneous analysis of highly polar compounds such as antibiotics (for Example, spiramycin, tilmicosin, gentamycin, spectinomycin, neomycin etc.), quinoxaline carboxylic acid and cyromazine. For simultaneous measurement of multiple drugs, it is necessary to simultaneously and equally extract plural residual veterinary drugs from livestock product or seafood. For veterinary drugs soluble in organic solvent, it has been known that the problem can be solved by using an organic solvent as an extracting solution (for Example, Howells, L, Sauer, M. J., Analyst, 126, 155-160, 2001). However, there are many problems in the extraction process, since organic solvent cannot be used for drugs hardly soluble in organic solvent.

Thus, in veterinary drugs soluble in organic solvent, the problem can be solved by using an organic solvent as an extracting solution. On the other hand, an extracting solution which can simultaneously extract plural water soluble veterinary drugs is also desirable. Such extracting solution has not been known yet, because the stable condition as medicines are different among antibacterial substances of veterinary drugs, and it is hard to simultaneously and efficiently extract plural residual drugs without degradation.

The present inventors repeated studies to solve this problem, and found out that plural residual veterinary drugs can be efficiently and simultaneously extracted without changing the properties of residual veterinary drugs by using an extracting solution having a specific nature. The invention depends on this finding, and it is hence an object of the invention to present a useful extracting solution to simultaneously extract residual veterinary drugs, especially antibacterial substances, in livestock product and seafood.

### DISCLOSURE OF THE INVENTION

To solve the problems, the present invention provides an extracting solution for use in extraction of residual veterinary drugs in livestock product or seafood comprising a buffer solution of pH 5.5 to 7.5 and containing 0.05 to 1% (w/v, same hereinafter) of metaphosphoric acid. In particular, the solution is preferably used in extraction of antibacterial substances, and the buffer solution is preferably McIlvaine buffer solution.

### BEST MODE FOR CARRYING OUT THE INVENTION

Veterinary drugs, particularly antibacterial substances, are difficult to simultaneously be extracted, because they are different in the stably-existing conditions. From such point of view, the inventors experimented as follows in order to overcome the problems.

First, sample substances to be tested were divided into two groups by polarity of an extracting solution. One group includes 21 compounds soluble in organic solvent such as tetracycline, and other group includes four compounds of aminoglycoside antibiotics having higher polarity and hardly soluble in organic solvent (for example, streptomycin, dihydrostreptomycin, spectinomycin and gentamycin).

A fortification recovery test was carried out by adding specified amounts of the sample substance to beef meat, extracting the sample with a mixed solvent of McIlvaine buffer solution (pH 7.0), EDTA and methanol, and refining with C₁₈ solid phase extraction column.

In 21 compounds of group 1, as a result of the fortification recovery test at a concentration of 20 ppb, the recovery rates and relative standard deviation conformed nearly to the standard values of the official compendium.

In aminoglycoside antibiotics of group 2, to adsorb these compounds on C₁₈ solid phase extraction column, heptafluorobutyric acid (HFBA) was added to an extractant solution (extracted supernatant) as an ion pair reagent, and the sample substance was extracted by C₁₈ solid phase extraction column. By this operation, streptomycin, dihydrostreptomycin, spectinomycin and gentamycin were anticipated to be recovered, but gentamycin was not recovered.

To clarify the cause, a standard substance was added to the extractant solution immediately before the treatment with C₁₈ solid phase extraction column, and then all the aminoglycoside antibiotics examined were recovered. It is hence known that the problem exists before the refining process by C₁₈ solid phase extraction column, that is, in the extraction process.

As a result of experiment by adding 1% metaphosphoric acid (pH 2) in the extracting solution as specified in the extraction condition of the official compendium for extracting gentamycin, gentamycin could be recovered. Hitherto, the 1% metaphosphoric acid solution (pH 2) was used in a deproteinizing operation, and the extracting solution was acidic because metaphosphoric acid was added.

When such extracting solution is used, unstable compounds such as erythromycin are decomposed in the acidic condition. Hence, an extracting solution of low pH cannot be used simultaneous analyses of multiple components. On the other hand, some compounds such as chlorotetracycline are unstable in alkaline condition, and it is necessary to extract in neutral condition to simultaneously measure multiple components. Studies were repeated to find out a better extraction method in neutral region.

First, in gentamycin extraction method, it was examined whether deproteinization in acidic condition is necessary or not. Because the metaphosphoric acid solution of the official compendium is pH 2, recovery of gentamycin was examined by using McIlvaine buffer solution in a pH range between 2 and 6, but the recovery of gentamycin was not recognized. Hence, the presence of metaphosphoric acid itself is concluded necessary for recovery of gentamycin.

Accordingly, as shown in Examples below, the inventors widely studied into liquid properties of an extracting solution and concentration of metaphosphoric acid, and discovered that veterinary drugs, especially, antibacterial substances can be simultaneously extracted without spoiling the stability, by using metaphosphoric acid at specific concentration in a neutral pH range, more specifically by using an extracting solution containing 0.05 to 1% of metaphosphoric acid and comprising a buffer solution of pH 5.5 to 7.5. The buffer solution containing metaphosphoric acid at such concentration and pH does not have deproteinizing effect as the purpose of use specified in the official compendium, and the existence of metaphosphoric acid itself is considered important for extraction effect.

In the invention, the buffer solution is not particularly specified as far as not affecting extraction of veterinary drugs from livestock product or seafood, and usable examples include McIlvaine buffer solution, HEPES buffer solution, Sorensen buffer solution, Clark-Lubs buffer solution, and Kolthoff's buffer solution, and McIlvaine buffer solution is particularly preferred.

To prevent degradation of veterinary drugs to be extracted, the pH of extracting solution of the invention is adjusted to 5.5 to 7.5, preferably 6.0 to 7.0.

Concentration of metaphosphoric acid is adjusted to 0.05 to 1%, preferably 0.1 to 0.5%. If concentration of metaphosphoric acid is less than 0.05%, gentamycin may not be extracted.

The extracting solution of the invention is prepared by adding metaphosphoric acid to the buffer solution, and adjusting to proper pH range and proper metaphosphoric acid concentration range mentioned above. At this time, to enhance the efficiency of extraction, as required, a chelating agent such as EDTA or a hydrophilic organic solvent such as methanol may be added. Preferably, an extracting solution contains buffer solution, chelating agent and methanol at a rate of about 15:1:4.

The extracting solution of the invention can be used same as the conventional extracting solution for residual veterinary drug, and for example, the extracting solution of the invention is added to a livestock or seafood sample and homogenized, and a supernatant is separated by conventional means such as centrifugal separation, and an extractant solution is obtained.

The amount of extracting solution to be used is adjusted to about 1:1 to 10 (w/v ratio, same hereinafter) in the ratio of the sample and the extracting solution, preferably 1:2 to 8, more preferably about 1:2.5 to 5. If the ratio of extracting solution is less than 1 to the sample, extraction may be insufficient, and there is no problem if exceeding 10, but the operation may be complicated, since the amount of extractant solution increases.

Livestock and seafood samples include livestock products (for example, beef, pork, chicken, rabbit, mutton, duck, etc.) and seafood (for example, young yellowtail, sea bream, blowfish, shrimp, eel, carp, trout, oyster, etc.). When investigating the environmental pollution by veterinary drugs, animals sampled from the nature may be examined.

The extractant solution obtained above is refined by a conventional method such as column refining, and the residual drugs are measured by using an ordinary apparatus.

In the refining method, for example, C₁₈ solid phase extraction column is used, and to adsorb on the column, at this time, an ion pair reagent such as HFBA may be also added to the extractant solution.

The residual veterinary drug may be measured by using high performance liquid chromatography and/or mass analysis method.

### INDUSTRIAL APPLICABILITY

By the extracting solution of the invention, since the pH of the solution is in neutral region, plural veterinary drugs can be extracted efficiency at the same time without spoiling the stability of the drugs. Since metaphosphoric acid is contained in the extracting solution, gentamycin can be also extracted. Therefore, by using the extracting solution of the invention, residual veterinary drugs in livestock product or seafood can be simultaneously extracted, and measurement of residual levels can be improved in terms of speed and simplicity of operation.

### EXAMPLES

The invention is more specifically described below by referring to Examples, but it must be noted that the invention is not limited to these Examples alone.

### Example 1

A fortification recovery test was conducted in the following method. The fortification recovery test of aminoglycoside compounds was carried out at a concentration of 200 ppb in consideration of residual standard values specified in the Japanese Food Hygiene Law.

Any one of standard samples of aminoglycoside antibiotics (streptomycin, dihydrostreptomycin, spectinomycin and gentamycin) was added at a concentration of 200 ppb, or not added (for Control) in 5 g of beef meat, which were homogenized and extracted in 25 ml of a mixed solvent of McIlvaine solution containing 0.1% metaphosphoric acid (pH 7), EDTA and methanol.

The extracted supernatant (extractant solution) was diluted by 25 ml of 10 mM HFBA, and applied to C₁₈ solid phase extraction column (12 cc, 500 mg), washed with 5 ml of 5 mM HFBA, and eluted with 20 ml of HFBA/MeOH (1:1).

The eluate was concentrated and dried, re-dissolved in a mixed solvent of DMSO/water (1.0 ml), and analyzed by LC/MS/MS under the following condition, and the recovery rate (%) was measured.

In a moving bed in a portion of high performance liquid chromatography, a gradient of solution A (0.3% acetic acid, 10 mM ammonium acetate) and solution B (acetonitrile containing 20% methanol) was used, and it was eluted at a flow rate of 220 μl/min by solid phase extraction column. Mass analysis was performed by an API2000 apparatus manufactured by Applied Biosystems, and the ionizing mode was carried out by using ESI positive and negative, in the conditions of spray voltage (pos.) of 5000, spray voltage (neg.) of −4200, and turbo gas temperature of 550° C.

### Example 2

Measurement was carried out in the same manner as in Example 1, except that McIlvaine solution containing 0.3% metaphosphoric acid (pH 6) was used for extraction, instead of McIlvaine solution containing 0.1% metaphosphoric acid (pH 6) as the extracting solution.

### Example 3

Measurement was carried out in the same manner as in Example 1, except that McIlvaine solution containing 0.5% metaphosphoric acid (pH 6) was used for extraction, instead of McIlvaine solution containing 0.1% metaphosphoric acid (pH 6) as the extracting solution.

### Example 4

Measurement was carried out in the same manner as in Example 1, except that McIlvaine solution containing 1% metaphosphoric acid (pH 6) was used for extraction, instead of McIlvaine solution containing 0.1% metaphosphoric acid (pH 6) as the extracting solution.

### Comparative Example 1

Measurement was carried out in the same manner as in Example 1, except that McIlvaine solution (pH 6) not containing metaphosphoric acid was used for extraction, instead of McIlvaine solution containing 0.1% metaphosphoric acid (pH 6) as the extracting solution.

### Comparative Example 2

Measurement was carried out in the same manner as in Example 1, except that McIlvaine solution containing 0.01% metaphosphoric acid (pH 6) was used for extraction, instead of McIlvaine solution containing 0.1% metaphosphoric acid (pH 6) as the extracting solution.

Test results are shown in Table 1. As shown in Table 1, in Example 1, the recovery rate in all steps was about 60% in dihydrostreptomycin, about 50% in streptomycin and spectinomycin, and less than 40% in gentamycin. Relative standard deviations of 4 specimens were the level of 10%, and a favorable repeatability was observed. In Example 2, Example 3 and Example 4, similar results were obtained. In Comparative Examples 1 and 2, recovery rates of gentamycin were too low to be used in measurement.

In Example 1, Example 2, Example 3 and Example 4, chromatograms obtained by LC/MS/MS were observed, almost no peak was detected in a meat extractant solution, and it was easy to detect the drugs and it was not confused by pseudopositive results. Since the baseline was low, lower limits of quantitation obtained by S/N were possible to about some tens of ppb.

TABLE 1

| | Example | Example | Example | Example | Comparative | Comparative |
|---|---|---|---|---|---|---|
| | 1 | 2 | 3 | 4 | Example 1 | Example 2 |
| Streptomycin | 49.3 | 52.2 | 54.1 | 55.3 | 43.4 | 25.1 |
| Dihydrostreptomycin | 59.2 | 60.1 | 61.2 | 62.1 | 45.3 | 28.5 |
| Spectinomycin | 51.5 | 51.4 | 51.0 | 50.9 | 72.3 | 17.9 |
| Gentamycin | 35.6 | 35.8 | 36.0 | 36.6 | 0.0 | 9.8 |

Thus, in aminoglycoside antibiotics, by adding metaphosphoric acid to the extracting solution at a lower concentration than conventional one, it is found that the compounds can be extracted in neutral pH region and the results are reproducibly obtained.

### Example 5

In 19 compounds soluble in organic solvent such as tetracycline in group 1, by the same method as Example 1, fortification recovery tests were carried out using a mixed solvent comprising McIlvaine solution containing 0.1% metaphosphoric acid (pH 7), EDTA and methanol as the extracting solution. Results are shown in Table 2.

As shown in Table 2, favorable extraction results were obtained in the substances of group 1.

Together with the results above, by using the extracting solution of the invention, it is confirmed that residual veterinary drugs can be widely extracted, and even high polar compounds such as antibiotics can be analyzed simultaneously.

TABLE 2

| | Compound name | Mean (%) |
|---|---|---|
| Cephems antibiotics | Cefazoline | 69.7 |
| Penicillins antibiotics | Nafcilin | 63.9 |
| | Benzyl penicillin | 74.8 |
| | Phenoxymethyl penicillin | 77.8 |
| Macrolides antibiotics | Erythromycin | 66.6 |
| | Oleandomycin | 67.3 |
| | Josamycin | 65.6 |
| | Spiramycin | 62.9 |
| | Neospiramycin | 51.6 |
| | Tilmicosin | 61.6 |
| | Tyrosine | 56.3 |
| Tetracyclines | Oxytetracycline | 66.0 |
| antibiotics | Tetracycline | 90.6 |
| | Chlorotetracycline | 88.9 |
| Others | Chloramphenicol | 64.9 |
| | Florfenicol | 59.3 |
| Lincosamides | Lincomycin | 73.5 |
| Dioxyquinoxzalines | Quinoxaline-2-carboxyic acid | 28.0 |
| | Cyromazine | 60.2 |

Note:
Mean is the average of 9 measurements.

1. An extracting solution for residual veterinary drugs in livestock product or seafood comprising 0.05% to 1% (w/v) of metaphosphoric acid and a buffer solution of pH 5.5 to 7.5.

2. The extracting solution according to claim 1, wherein the veterinary drugs are antibacterial substances.

3. The extracting solution according to claim 2, wherein the antibacterial substances are aminoglycoside antibiotics.

4. The extracting solution according to any one of claims 1 to 3, wherein the buffer solution is McIlvaine buffer solution.

5. The extracting solution according to claim 2, wherein the antibacterial substances are antibiotics selected from the group of consisting of Cephems, Penicillins, Macrolides, Tetracyclines, Lincosamides, Chloramphenicol, Florfenicol and Dioxyquinoxzalines.

6. A method for measuring residual veterinary drugs in a livestock product, or seafood sample, comprising:
contacting said sample with the extracting solution of claim 1 under conditions wherein residual veterinary drugs are extracted from said sample to prepare an extract; and
measuring residual veterinary drugs in said extract.

7. The method according to claim 6, wherein the sample is a livestock product sample selected from the group consisting of beef, pork, chicken, rabbit, mutton and duck.

8. The method according to claim 6, wherein the sample is a seafood sample selected from the group consisting of young yellowtail, sea bream, blowfish, shrimp, eel, carp, trout and oyster.

9. The method according to claim 6, wherein said sample is obtained from one or more animals from nature in order to investigate residual veterinary drug environmental pollution.

10. A method for measuring veterinary drugs remaining in a livestock product or seafood comprising:
a) preparing the extracting solution according to claim 1 by adding metaphosphoric acid to a buffer solution, adjusting pH within the range of 5.5 to 7.5, adjusting the metaphosphoric acid concentration within the range 0.05% to 1% (w/v) and adding a chelating agent or a hydrophilic organic solvent to the extracting solution;
b) weighing out a sample of a livestock product or seafood;
c) adding said extracting solution to said sample;
d) adjusting the volume of said extracting solution to the weight of said sample in a ratio of 1:1 to 10;
e) grinding or homogenizing said sample and said solution into a homogenous mixture;
f) filtering or centrifuging said mixture to recover said extracting solution;
g) refining recovered said extracting solution by adding an ion pair reagent to said extracting solution, and subsequently charging said extracting solution to a solid phase extraction column;
h) eluting residual veterinary drugs by washing said extraction column with additional ion pair reagent in an eluent;
i) concentrating the residual veterinary drugs within said eluent by drying, then subsequently re-dissolving said residual veterinary drugs within a buffer for analysis; and
j) measuring residual veterinary drugs within said sample by high performance liquid chromatography and/or mass spectral analysis.

11. The method according to claim 10, wherein in step d) the adjusted volume of said extracting solution to the weight of said sample is in a ratio of 1:2.5 to 5.

12. The method according to claim 10, wherein in step a) said extracting solution contains buffer solution, chelating agent and methanol at a ratio of about 15:1:4.
